(19) United States
(12) Patent Application Publication
(10) Pub. No.: US 2002/0187180 A1
(43) Pub. Date: Dec. 12, 2002

(54) Modifying undesirable tastes

(76) Inventors: Gary J. Calton, Elkridge, MD (US); Louis L. Wood, Rockville, MD (US)

Correspondence Address: Beverly J. Artale, Suite 001, 3826 Sunflower Circle, Mitchellville, MD, 20721, US

(21) Appl. No.: 10/172,117

(22) Filed: Jun. 14, 2002

Related U.S. Application Data
(63) Continuation of application No. 09/517,257, filed on Mar. 2, 2000, now abandoned.

(51) Int. Cl. A61K 31/737; A23G 3/00

(52) U.S. Cl. 424/439; 514/54; 426/658

(57) ABSTRACT

A method of inhibiting an undesirable taste in oral compositions such as foods, beverages, and pharmaceuticals is disclosed. Also disclosed are oral and pharmaceutical compositions comprising undesirable tasting compounds wherein said undesirable tastes are inhibited by the addition of at least one sulfated polysaccharide to the oral and pharmaceutical compositions. Preferred sulfated polysaccharides include carrageenan compounds or a mixture thereof.

### TECHNICAL FIELD

The present invention relates to the reduction of undesirable tastes in oral compositions. More particularly, this invention relates to novel oral compositions having at least one unpleasant tasting component, wherein said compositions contain at least one sulfated polysaccharide component as an agent for reducing or inhibiting the taste of said unpleasant tasting component. The present invention also relates to a method of inhibiting undesirable tastes in orally administered compositions by the addition of at least one sulfated polysaccharide compound in an amount sufficient to eliminate or reduce said undesirable tastes.

### BACKGROUND OF THE INVENTION

Consumers do not care for unpleasant tastes such as bitter and metallic tastes in the broadest sense. The desire for improved palatability of oral compositions having unpleasant tasting components has prompted the development of numerous formulations and methods of removing undesirable tastes in orally adminsterable compositions.

In most cases, reduction of unwanted or unpleasant tastes in oral compositions has heretofore involved the addition of a masking compound, such as flavors and sugars or other sweetening ingredients, to mask the unwanted tastes. For example, compounds conventionally used to mask bitter flavors in oral compositions have included, inter alia, phosphorylated amino acids (U.S. Pat. No. 5,766,622); gelatin (Japanese Patent Application No. 04-346,937); gelatinized starch (Japanese Patent Application No. 04-235,136); acidic amino acids (U.S. Pat. No. 4,517,379); chitosan (Japanese Patent Application No. 04-009,335); cyclodextrins (U.S. Pat. No. 5,024,997); liposomes (U.S. Pat. No. 5,009,819); lecithin or lecithin like substances (Japanese Patent Application No. 62-265,234); surfactants (U.S. Pat. No. 5,439,671); salts (U.S. Pat. No. 5,262,179); and the like.

Attempts to mask unpleasant tastes in oral compositions have also included such techniques as coating or microencapsulation (European Patent Application No. 551,820); functional group alteration (U.S. Pat. No. 5,350,839); and structural matrix forms of taste masking have been used. Oral compositions employing such technology have incorporated agents such as silicate clays (U.S. Pat. Nos. 3,140,978 and 4,581,232); acrylic acid copolymers (U.S. Pat. No. 5,286,489); gums (U.S. Pat. No. 5,288,500); cellulose (U.S. Pat. No. 5,192,563); and waxes in an effort to provide improved tasting compositions.

In many cases, masking has proven ineffective to remove unpleasant tastes in oral compositions. Consequently, other methods of inhibiting or reducing bitter tastes have been developed. For example, Kurtz and Fuller (U.S. Pat. Nos. 5,232,735, and 6,008,250) have disclosed modifying certain compounds to block the taste of an undesirable tasting component contained in an oral composition. Roy et al. (U.S. Pat. Nos. 4,994,490 and 5,266,717) have disclosed N-(sulfomethyl)-N′-arylureas as sweetness and bitterness inhibitors. Guadagni et al. (U.S. Pat. No. 4,154,862) have found that the addition of the flavone, neodiosmin, results in reduced bitterness and aftertaste, while Riemer (U.S. Pat. No. 5,336,513) has discovered that certain cinnamic acid derivatives have the ability to inhibit the taste of bitter compounds and the aftertaste of artificial sweeteners. Magnolato (U.S. Pat. No. 4,282,264) has disclosed a process for removing bitter taste from fruit and vegetable extracts by selective absorption using a ligneous material and Miller (U.S. Pat. No. 4,248,141) disclosed using steam to remove the bitter taste from soybeans. Buist (U.S. Pat. No. 5,411,757) has masked the bitter tastes of the amino acids by omitting certain unpalatable amino acids from the formula or acetylating the amino acids to reduce their unpalatability.

The solution to reduction of unwanted tastes in orally ingestible compositions would ideally involve the development of an inhibitor which provides a neutral flavor to the compositions. The compositions may thereafter be flavored to suit. Katsuragi and Kurihara have reported in Nature, vol. 365, pp. 213-214 (1993), a bitterness inhibitor made of phosphatidic acid and beta-lactoglobulin, which inhibitor suppresses taste responses and sensations to bitter substances without affecting the responses to other taste stimuli. This compound has, however, shown only limited scope to inhibit bitter tasting compounds in oral compositions.

Consequently, there exists a need in the industry for a universal masking agent which is effective to remove a variety of unwanted tastes occurring in various foods, beverages, and pharmaceutical preparations and which does not compromise the taste quality of the preparations.

### SUMMARY OF THE INVENTION

We have now discovered that the addition of specified amounts of at least one sulfated polysaccharide to oral compositions, such as foods, beverages, and pharmaceuticals, unexpectedly inhibits a variety of unwanted tastes. The sulfated polysaccharides do not dilute the undesirable tastes, but effectively masks the tastes while providing a neutral flavor to the treated compositions. The treated compositions may thereafter be flavored with conventional flavoring agents.

Accordingly, an advantage of the present invention is to provide a method for inhibiting undesirable or unwanted tastes in oral compositions, such as foods, drinks, over-the-counter and prescription pharmaceuticals, and toiletries, by adding to the composition at least one sulfated polysaccharide in an amount sufficient to reduce or inhibit the oral perception of the undesirable or unwanted taste.

It is also an advantage of the present invention to provide a method of inhibiting undesirable tastes in such oral compositions while leaving a neutral flavor to the compositions and permitting the neutral compositions to be flavored using conventional flavoring components.

It is a still further advantage of the present invention to provide novel oral compositions, such as foods, drinks, over-the-counter and prescription pharmaceuticals, and toiletries, comprising an undesirable tasting component and at least one sulfated polysaccharide in an amount sufficient to mask the taste of said undesirable component.

It is also an advantage of the present invention to provide pleasant tasting oral compositions, such as foods, drinks, over-the-counter and prescription pharmaceuticals, and toiletries, containing at least one unpleasant tasting component, in particularly, at least one unpleasant tasting amino acid.

It is yet another advantage of the present invention to provide pleasant tasting pharmaceutical compositions having an undesirable tasting component and at least one sulfated polysaccharide in an amount sufficient to mask the taste of said undesirable component.

It is yet another advantage of the present invention to provide pleasant tasting pharmaceutical compositions for treating cough/cold symptoms comprising at least one pharmacologically active cough or cold relieving or reducing agent having an undesirable taste and at least one sulfated polysaccharide in an amount effective to mask the undesirable taste of said agent.

It is a still further object of the present invention to provide pleasant tasting oral compositions for relief of gastrointestinal distress, which compounds comprise a component having a bitter and/or metallic taste and at least one sulfated polysaccharide in an amount effective to mask the bitter and/or metallic taste.

These and other advantages of the present invention will become readily apparent from the detailed description and the claims which follow.

### DETAILED DESCRIPTION OF THE INVENTION

In practicing the present invention, at least one sulfated polysaccharide is added to an oral composition having at least one undesirable tasting component, in an amount sufficient to mask unpleasant tastes associated with said component. The phrase “oral composition/s”, as used herein, is defined as any product, i.e. foods, beverages, over-the-counter and/or prescription pharmaceuticals, toiletries and the like, which in the ordinary course of usage is intentionally ingested orally into the body of a human or animal.

The phrase “undesirable or unwanted taste”, as used herein, is not limited by the basic tastes of sweet, sour, bitter, umami, and salty; but is defined as any taste, including sweet, bitter, sour, alkaline, astringent, tangy, dry, sharp, cool, hot, burning, acidic, spicy, pungent, woody, smoky, umami, metallic, and/or any aftertaste, if such taste is unwanted in a composition.

The term “inhibit”, as used herein, is defined as the slowing of or interference in the taste transduction mechanism such that, while an undesirable tasting component remains chemically unaltered within a composition, other than salt or ion formation, the perception of the undesirable taste of the compound is decreased in the person or animal consuming said composition.

The term “mask”, as used herein, is defined as the addition of a material to compositions having an undesirable tasting component, which material does not chemically alter the undesirable tasting component contained in the compositions, other than salt or ion formation, but acts to cover, disguise, and/or obscure the taste of the component such that perception of the undesirable taste by a human or animal consuming said composition is inhibited, reduced or eliminated.

The terms “chemical alter” and/or “chemically unaltered”, as used herein, are defined as the structural modification of a chemical compound, other than salt or ion formation.

The term “compatible” is used herein to mean that the components of the compositions are capable of being physically mixed or co-mingled with one another without substantially reducing the efficacy of the components or the composition under ordinary use conditions.

The term “sulfated polysaccharide” is used herein to mean compound containing at least one polymeric sugar moiety covalently attached to a sulfate group. One example of a sulfated polysaccharide is the carrageenan class of compounds. Other examples of sulfated polysaccharides include chondroitin sulfate, sulfated cyclodextrins, dextran sulfate and heparin sulfate.

The term “fat” is used herein to mean a fat derived from a vegetable or animal, whether solid or liquid in its purified form. Such materials are also referred to as oils.

The term “amino acid”, as used herein, is defined as an organic acid which has an amine in the alpha position, either in the D or L form, which may be used in an oral composition. Such amino acids used in human nutrition commonly contain mixtures of varying amounts of some or all of the following amino acids: glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-ornithine, L-proline, L-serine, L-threonine, L-tryptophan, L-tyrosine, L-valine, and D,L-methionine.

In accordance with the present invention, sulfated polysaccharide compounds are added to oral compositions having at least one unpleasant tasting component to inhibit or eliminate a variety of unwanted or undesirable tastes in the compositions. It has been found that the sulfated polysaccharides of this invention are especially useful in the inhibition of the undesirable tastes of compounds having an amine group. Although not wishing to be bound by any particular theory, it is speculated by the inventors that the sulfate groups are especially reactive in forming salts with the amine groups of the undesirable tasting ingredients in the pharmaceuticals, beverages, toiletries, and foods having such undesirable tastes, thus preventing the undesirable taste which is postulated to be due to the amine groups. The acid groups are thereafter easily masked and are even desirable at times due to the pleasant taste of acids in oral compositions such as food, beverages, pharmaceuticals and toiletries.

Any sulfated polysaccharide will be useful and intended within the scope of the invention provided that it is compatible with the active and essential components comprising the oral compositions. The use of sulfated polysaccharides in the present invention is especially advantageous as many of these compounds are classified by the FDA as “Generally Recognized as Safe” having been used in foods, beverages, pharmaceuticals and toiletries for years at lower levels than those of the present invention and for the different purposes of water and fat stabilization, gelling, thickening, emulsifying and smoothing the texture of pharmaceuticals, beverages, toiletries, and foods.

A preferred sulfated polysaccharide is a carrageenan or mixture of carrageenans. Suitable carrageenans include, but are not limited to, iota, kappa and lambda carrageenans and mixtures thereof. In a more preferred embodiment the sulfated polysaccharide is iota or kappa carrageenan. In a still more preferred embodiment the sulfated polysaccharide is lambda carrageenan.

Carrageenans are sulfated carbohydrates which are obtained mainly from red algae by extraction in the presence of lime (see for instance U.S. Pat. No. 3,956,173). In addition, carrageenans are widely available from various commercial sources such as FMC, Chicago, Ill., Colliodes Naturel, Bridgewater, N.J. or Integrated Solutions Inc., Searsport, Me.

Unless otherwise noted, the carrageenans used in accordance with the present invention are used in the form their sodium, potassium, and calcium salts of the sulfate groups or mixtures thereof. It is, however, possible to use the carrageenans as their acid sulfate forms free or partially free of Na, K, and Ca ions. Solutions of the acid sulfate forms of the carrageenans are obtained by washing away the Na, K, and Ca ions with aqueous solutions of strong acids such as HCl, HNO₃, H₂SO₄ and the like.

Other examples of sulfated polysaccharides includebut are not limited to, chondroitin sulfate, sulfated cyclodextrins, dextran sulfate, heparin sulfate and the like.

In accordance with the invention, the amount of at least one sulfated polysaccharide to be used in accordance with the present invention is any amount sufficient to inhibit or reduce the oral perception of an undesirable tasting component without substantially reducing the efficacy of the components or the composition under ordinary use conditions. As would be understood by the skilled artisan, the optimum concentration of sulfated polysaccharide compound will vary depending upon such factors as the nature of the composition, the nature of the undesirable tasting component/s, the concentration of the component/s having the undesirable taste, the degree of inhibition desired and the compatibility of the sulfated polysaccharide with the component/s of the composition to be treated. Such an optimum concentration is readily determined by the skilled artisan by conducting routine sensory experiment.

In any case, the level of sulfated polysaccharide substantially exceeds amounts heretofore used in foodstuffs as thickening and smoothing additives, e.g. typically, 0.03% to 0.04% for iota carrageenan in milk products (Shemberg Product Data Sheet, Benlacta S-100, Shemberg USA, Searsport, Me.); 0.1 to 0.2% for lambda carrageenan in milk products (Shemberg Product Data Sheet, Isovis CS-9314, Shemberg USA, Searsport, Me.); and 0.4 to 1% for kappa carrageenan in meat products (Liangel F, Colloides Naturels, Inc., Bridgeport, N.J.).

For example, to mask a bitter/metallic taste or aftertaste a sulfated polysaccharide is added to food, pharmaceutical and toiletry compositions in an amount ranging from about 2% to about 97% by weight of the formulated end product. Preferably, the sulfated polysaccharide is added in an amount greater than about 2% to about 95% by weight of the composition. Most preferably, the sulfated polysaccharide is added in an amount greater than about 5% to about 92% by weight of the composition.

To mask a bitter/metallic taste or aftertaste in a beverage composition, a sulfated polysaccharide is added in an amount ranging from about 0.5% to about 20% by weight of the formulated end product. Preferably, the amount of sulfated polysaccharide to be added is greater than about 0.75% to about 10% by weight of the beverage composition. Most preferably, the amount of sulfated polysaccharide added is greater than about 1% to about 5% by weight of the beverage composition.

The sulfated polysaccharide is added to foods, beverages, pharmaceutical and other oral compositions to inhibit or suppress sweet, bitter, sour, salty, alkaline, astringent, tangy, sharp, acidic, spicy, pungent, woody, smoky, umami, metallic, any aftertaste, and mixtures thereof.

Examples of foods having an undesirable or unwanted taste include but are not limited to, citrus fruits such as grapefruit, orange, and lemon; vegetables such as tomato, pimento, celery, melon, carrot, potato and asparagus; seasoning or flavoring materials, soy sauce, red pepper, soybean products, fish products, meats and processed meats; dairy products such as cheese; breads and cakes, confectioneries such as candies, chewing gum and chocolate and specifically prepare foods for health.

Examples of drinks having an undesirable or unwanted taste include, but are not limited to, juices of citrus fruits and vegetables, soybean, milk, coffee, cocoa, black tea, green tea, fermented tea, semi-fermented tea, refreshing drinks, beverages and milk.

In a preferred embodiment of this invention, the sulfated polysaccharides are useful to inhibit the taste of pharmacologically active ingredients having an undesirable or bitter/metallic taste in pharmaceutical compositions. Examples of pharmaceutical compositions comprising pharmacologically active compounds having an undesirable or bitter/metallic taste components include, but are not limited to, compositions useful for treating cough, cold, cold-like, allergy and/or flu symptoms and gastrointestinal distress. Such actives may be selected from, but are not limited to, a pharmacologically active having analgesic, anti-inflammatory, antipyretic, anesthetic, antihistamine, bronchodilators, decongestant, cough suppressants, demulcents, antitussives, and/or expectorant properties. The sulfates polysaccharides may also be added to compositions comprising such pharmacologically actives such as laxatives, antidiarrheals, anorexiants, anticholinergics, and antinauseants.

The undesirable taste of other basic pharmacologically active acid addition salts such as strychnine, quinine, papaverine, berberine, promethazine, brucine, propranolol, and chlorpromazine may also be suppressed by the addition of at least one sulfated polysaccharide.

Such pharmacologically actives are used in pharmaceutical compositions in accordance with the invention in conventional acceptable dosage ranges and carriers as is well known and easily determinable by one skilled in the pharmaceutical art.

In a particular preferred embodiment, sulfated polysaccharide compounds are useful to inhibit the undesirable tastes of components in a nutriceutical composition. Examples of nutriceutical compositions having an undesirable or bitter/metallic taste include enteral nutrition products for treatment of nutritional deficit, trauma, surgery, Crohn's disease, renal disease, hypertension, obesity and the like, to promote athletic performance, muscle enhancement or general well being or inborn errors of metabolism such as phenylketonuria.

In particular, such nutriceutical formulations may contain one or more amino acids which have a bitter or metallic taste or aftertaste. Such amino acids include, but are not limited to, an essential amino acids selected from the group consisting of L isomers of leucine, isoleucine, histidine, lysine, methionine, phenylalanine, threonine, tryptophan, tyrosine and valine, including functional analogs of methionine, such as hydroxy methionine or D,L-methionine.

In accordance with the invention. the sulfated polysaccharide may be incorporated into foods, beverages or pharmaceutical compositions using conventional blending and mixing techniques. Mixtures of two or more sulfated polysaccharides may be optionally employed. Final compositions comprising the sulfated polysaccharide additives may be in any form such as solid or semi-solid preparations (e.g., gums, custards, foods, capsule, granules, medicinal pill, powder, pellet, troche and dry syrup), and liquid preparations (e.g., liquids, gels, extracts, elixirs, spirits, syrups, aromatic water, lemonades, and fluid-extracts). The sulfated polysaccharide additives can be incorporated into the oral preparation singly or in combination with one or more of known additives. Examples of such known additives include, but are not limited to, diluents, filler, recipient, vehicle, binder, disintegrator, lubricant, fluidity-improving agent, coating agent, flavor, masking agent, perfume, anti-oxidation agent and the like.

The sulfated polysaccharides may also be coated onto a composition having an unpleasant taste. For instance, foods in the form of a solid, such as candy, confectioneries, processed fish/meats, etc. or pharmaceuticals in the form of powder, granules, pellets, tablets, soft and hard capsules and pills. The coating layer may comprise the sulfated polysaccharide and hydrophilic polymers such as cellulose derivatives, gelatin and polyvinyl alcohol. Other additives such as sweeteners and flavors may be incorporated into the coating layer.

### EXAMPLES

The following examples further describe and demonstrate embodiments within the scope of the present invention. These examples are given solely for the purpose of illustration and are not to be construed as a limitation of the present invention as many variations thereof are possible without departing from the spirit and scope of the disclosed invention. All percentages and ratios used herein are by weight and all measurements made at 25° C., unless otherwise specified.

### Example 1

2 grams of iota carrageenan was added to a mixture of amino acids consisting of L-histidine, 7.97%; L-isoleucine, 10.14%; L-leucine, 15.94%; L-lysine, 11.59%; L-methionine, 15.94%; L-phenylalanine, 15.94%; L-threonine, 7.25%; L-tryptophan, 3.62%; and L-valine, 11.59%; to provide three carrageenan/amino acid mixtures having 25%, 16% and 9% amino acid content (i.e., 75%, 84% and 91% carrageenan content), respectively, prior to the addition of water. The ingredients of each carrageenan/amino acid mixture were mixed together and then 10 g of boiling water was added with rapid mixing.

The products were taste tested and scored on the basis of taste. Using 10 times the amount of water or half the amount of water did not change the taste scores. A score of 10 (a bitter metallic taste) to 1 (a bland non-bitter, non-metallic taste) was given depending upon taste. Results are recorded in Table 1 below.

TABLE 1

| Amino Acid Content | iota Carrageenan | |
|---|---|---|
| (Prior to water addition) | Concentration | Taste |
| 25% | 75% | 9 |
| 16% | 84% | 6 |
| 9% | 91% | 1 |

At 75% iota carrageenan content, the bitter/metallic taste and aftertaste was slightly ameliorated while at 84%, it was greatly ameliorated and at 91% carrageenan content, the bitter/metallic taste and aftertaste was nearly completely eliminated.

### Example 2

Example 1 was repeated except the carrageenan/amino acid mixture was heated for 10 minutes at 95° C. after mixing was completed. Results of the taste test are recorded in Table 2 below:

TABLE 2

| Amino Acid Content | Iota Carrageenan | |
|---|---|---|
| (Prior to water addition) | Content | Taste |
| 16% | 84% | 1 |
| 9% | 91% | 1 |

At both 84% and 16% carrageenan content, the bitter/metallic taste and aftertaste was greatly improved, showing that heating improved the taste. It also improved the gel strength.

### Example 3

The procedure of Example 1 was repeated except that kappa carrageenan was used instead of iota carrageenan at the indicated quantity and the mixture, both with and without additional heating after mixing. Results are recorded in Table 3 below.

TABLE 3

| Amino Acid Content | Kappa Carrageenan | |
|---|---|---|
| (Prior to water addition) | Content | Taste |
| 16% | 84% | |
| heated to gel | | 2 |
| unheated | | 3 |
| 9% | 91% | |
| heated to gel | | 1 |
| unheated | | 3 |

Although slightly inferior to iota carrageenan, the kappa carrageenan was effective at masking the bitter/metallic taste and aftertaste of the amino acid components in the mixture.

### Example 4

14 g of gelatin powder was added to a solution of 6.7g of the amino acids mixture used in Example 1 in 400 ml water at 25° C. with stirring. The mixture was boiled for 5 minutes with stirring. Cooling the resultant solution to 25° C. gave a clear gel. The score of the gel was 10 on the taste test as no taste improvement was noted in the gelatin gel.

This shows the effect of the sulfated polysaccharide in ameliorating the taste of bitter compounds as exemplified in Examples 1-3 and 5-14, is not just a dilution effect, but a true inhibition or masking of the bitter/metallic taste of the bitter/metallic tasting components in the mixture.

### Example 5

1.0 g of the initial amino acid mixture as described in Example 1 was mixed with 2.0 g lambda carrageenan to obtain mixture having a 33% amino acid concentration. Thereafter 20g of water heated at 95° C. was added after which the mix was vigorously stirred. Upon tasting according to the scale in Example 1, the taste rating was 1. Thus, lambda carrageenan is very effective in inhibiting the bitter/metallic taste of amino acid.

### Example 6

1.0 g of an amino acid mixture containing an amino acid analog, hydroxymethylthiobutyrate (said mixture consisting of L-histidine, 12.25%; L-lysine, 34.04%; hydroxymethylthiobutyrate, 19.21%; L-threonine, 17.25%; L-tryptophan, 7.56%; and L-tyrosine, 9.68%), was mixed with 0.5 g lambda carrageenan to obtain a carrageenan/amino acid mixture having 67% amino acid and 33% carrageenan. 20 g of water heated at 95° C. was added after which the carrageenan/amino acid mixture was vigorously stirred. Upon tasting according to the scale in Example 1, the taste rating was 3. Thus, lambda carrageenan is very effective in inhibiting the bitter/metallic taste and aftertaste of the amino acid components in the mixture, many of which components are known to have a bitter metallic aftertaste.

### Example 7

A cough formula containing 100 mg of guaifenesin in 5 mL of formula which also contained caramel, citric acid, FD&C Red 40, flavors, glucose, glycerin, high fructose corn syrup, saccharin sodium, sodium benzoate and water was judged to have a very unpleasant bitter flavor even though it was a commercially available formula. The addition of 100 mg of lambda carrageenan with rapid mixing resulted in a formula that had virtually no bitter taste.

### Example 8

A cough, nasal decongestant, expectorant formula containing 100 mg of guaifenesin, 12.5 mg of phenylpropanolamine hydrochloride and 10 mg of dextromethorphan hydrobromide in 5 mL of formula which also contained caramel, citric acid, FD&C Red 40, flavors, glycerin, propylene glycol, saccharin sodium, sodium benzoate, sorbitol and water was judged to have a very unpleasant bitter flavor even though it was a commercially available formula. The addition of 200 mg of lambda carrageenan with rapid mixing resulted in a formula that had virtually no bitter taste.

### Example 9

A cough, nasal decongestant, expectorant formula containing 100 mg of guaifenesin and 10 mg of dextromethorphan hydrobromide in 5 mL of formula which also contained aspartame, benzoic acid, flavors, glycerin, hydroxyethyl cellulose, menthol, propylene glycol, sorbic acid and water was judged to have a very unpleasant bitter flavor even though it was a commercially available formula. The addition of 200 mg of lambda carrageenan with rapid mixing resulted in a formula that had virtually no bitter taste.

### Example 10

A cough, nasal decongestant, expectorant formula containing 15 mg of dextromethorphan hydrobromide in 5 mL of formula which also contained alcohol, citric acid, FD&C Red 40, flavors, high fructose corn syrup, glucose, glycerin, saccharin sodium, sodium benzoate and water was judged to have a very unpleasant bitter flavor even though it was a commercially available formula. The addition of 200 mg of lambda carrageenan with rapid mixing resulted in a formula that had virtually no bitter taste.

### Example 11

A drink was prepared by mixing a group of amino acids (3.5 g of the amino acid mixture of Example 1 with the addition of tyrosine to bring the level of tyrosine to 10.35%) with 3.5 g of a sulfated polysaccharide (lambda carrageenan) and 250 mL of 25° C. water (concentration of amino acids was 13.6 g/L, and the carrageenan level was 13.6%), lemon flavor, aspartame, citric acid, vitamin C, calcium carbonate and vitamin E in oil. The drink had a smooth, creamy, lemon flavor with no aftertaste. The same drink made without the sulfated polysaccharide was very undesirable in taste and left a bitter metallic aftertaste.

### Example 12

A mixture of 1.0 g lambda carrageenan and 2.0 g of iota carrageenan was thoroughly blended (2 minutes of high shear stirring) with a solution of 0.5 g of the amino acids mixture described in Example 1 in 20 ml of 80° C. water. The resultant homogeneous paste was pressed into a ⅛″ thick slab between two polyethylene films. The covered slab was baked 30 minutes at 70-90° C. for 30 minutes to give a flexible gel which was sliced into ½″ wide noodles having no bitter taste. The noodles were dried in air at 70-100° C. for 60 minutes to give hard brittle ¼″×1/16″ noodles. Treating these noodles with 50 ml 100° C. water for 3 minutes gave soft flexible ½″×¼″ noodles having no bitter taste. It is contemplated that various flavors (beef, chicken, shrimp, and the like) can be added to the aqueous noodle mix to give interesting food compositions.

### Example 13

A homogeneous paste of the carrageenans, amino acids, and water was prepared as described in Example 12. The paste was placed in a 50 ml syringe having a ¼″ orifice. The paste was extruded as a continuous ¼″ diameter noodle onto a polyethylene sheet. The noodle was dried in air at 70-100° C. for one hour to give a ⅛″ diameter hard brittle noodle. The dried noodle after 3 minutes in 100° C. water converted to a soft flexible noodle having no bitter taste. Again, as in Example 12, the addition of various flavors would give interesting food compositions.

### Example 14

A homogeneous blend of 1.0 g of lambda carrageenan, 2.0 g iota carrageenan, 2.0 g cellulose fiber (Niche Pharmaceuticals, Inc., Unifiber), and 1.0 g olive oil was thoroughly mixed (2 minutes high shear stirring) with a solution of 0.5 g of the amino acids mix (Example 1) in 40 ml 80 C water to give a homogeneous paste. This paste was pressed between two polyethylene films to give a ⅛″ thick slab. The covered slab was baked at 70-90° C. for 30 minutes to give a flexible gel having the feel and texture of cheese and no bitter taste. Again, as in Example 12, the addition of various flavors would give interesting food compositions.

We claim:

1. A method of inhibiting an undesirable taste in a oral composition comprising adding to an orally administrable composition having at least one component having an undesirable taste, a sulfated polysaccharide in an amount effective to inhibit all or a portion of the undesirable taste.

2. The method of claim 1 wherein the sulfated polysaccharide is a carrageenan.

3. The method according to claim 2 wherein said carrageenan is selected from the group consisting of iota carrageenan, kappa carrageenan, lambda carrageenan and mixtures thereof.

4. The method according to claim 1 wherein said orally administrable composition is selected from the group consisting of foods, beverages, pharmaceuticals, nutriceutical, toiletries and mixtures thereof.

5. The method according to claim 1 wherein said undesirable taste is selected from the group consisting of sweet, bitter, sour, salty, alkaline, astringent, tangy, sharp, acidic, spicy, pungent, woody, smoky, umami, metallic, an aftertaste, and mixtures thereof.

6. The method according to claim 5 wherein said undesirable taste is selected from the group consisting of bitter, metallic, and mixtures thereof.

7. The method according to claim 2 wherein said component having an undesirable taste comprises at least one amino acid.

8. The method of claim 7 wherein the concentration of said at least one amino acid present in the orally administrable composition is at least 0.1 g per 100 g of said composition.

9. The method of claim 8 wherein said carrageenan is present in a ratio of at least 0.5 part carrageenan to 1 part amino acid present in the orally administrable composition.

10. The method of claim 8 wherein said carrageenan is present in a ratio of at least 0.8 part carrageenan to 1 part amino acid present in the orally administrable composition.

11. The method of claim 1 wherein said component having an undesirable taste comprises at least one pharmacologically-active ingredient selected from the group consisting of bronchodilators, anorexiants, antihistamines, nutritional supplements, laxatives, analgesics, anesthetics, antacids, H2-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof.

12. The method of claim 2 wherein said carrageenan compound is an iota carrageenan.

13. The method of claim 2 wherein said carrageenan is kappa carrageenan.

14. The method of claim 2 wherein said carrageenan is lambda carrageenan.

15. The method of claim 7 wherein said at least one amino acid is selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine and mixtures thereof.

16. The method of claim 1 wherein said orally administrable composition is optionally heated following addition of the sulfated polysaccharide.

17. An orally administrable composition comprising at least one component having an undesirable taste or aftertaste and a sulfated polysaccharide in an amount sufficient to inhibit all or a portion of said undesirable taste or aftertaste.

18. The composition of claim 17 wherein the sulfated polysaccharide is a carrageenan.

19. The composition of claim 18 wherein said carrageenan is selected from the group consisting of iota carrageenan, kappa carrageenan, lambda carrageenan and mixtures thereof.

20. The composition of claim 17 wherein said orally administrable composition is selected from the group consisting of foods, beverages, pharmaceuticals, nutriceutical, toiletries and mixtures thereof.

21. The composition of claim 17 wherein said undesirable taste is selected from the group consisting of sweet, bitter, sour, salty, alkaline, astringent, tangy, sharp, acidic, spicy, pungent, woody, smoky, umami, metallic, an aftertaste, and mixtures thereof.

23. The composition of claim 17 wherein said undesirable taste is selected from the group consisting of bitter, metallic, and mixtures thereof.

24. The composition of claim 18 wherein said component having an undesirable taste comprises at least one amino acid.

25. The composition of claim 24 wherein the concentration of said at least one amino acid present in the orally administrable composition is at least 0.1 g per 100 g of said composition.

26. The composition of claim 24 wherein said carrageenan is present in a ratio of at least 0.5 part carrageenan to 1 part amino acid present in the orally administrable composition.

27. The composition of claim 25 wherein said carrageenan is present in a ratio of at least 0.8 part carrageenan to 1 part amino acid present in the orally administrable composition.

28. The composition of claim 17 wherein said component having an undesirable taste comprises at least one pharmacologically-active ingredient selected from the group consisting of bronchodilators, anorexiants, antihistamines, nutritional supplements, laxatives, analgesics, anesthetics, antacids, H2-receptor antagonists, anticholinergics, antidiarrheals, demulcents, antitussives, antinauseants, antimicrobials, antibacterials, antifungals, antivirals, expectorants, anti-inflammatory agents, antipyretics, and mixtures thereof.

29. The composition of claim 18 wherein said carrageenan compound is an iota carrageenan.

30. The composition of claim 18 wherein said carrageenan is kappa carrageenan.

31. The composition of claim 18 wherein said carrageenan is lambda carrageenan.

32. The composition of claim 24 wherein said at least one amino acid is selected from the group consisting of glycine, L-alanine, L-arginine, L-aspartic acid, L-cystine, L-glutamic acid, L-glutamine, L-histidine, L-isoleucine, L-leucine, L-lysine, L-methionine, L-ornithine, L-phenylalanine, L-proline, L-serine, L-threonine, L-tryptophan, L-tyrosine, L-valine, creatine and mixtures thereof.

33. A pharmaceutical composition comprising at least one orally administratable pharmacologically active component having an undesirable taste or aftertaste, and a sulfated polysaccharide in an amount sufficient to inhibit all or a portion of said undesirable taste or aftertaste.

34. The composition of claim 33 wherein the sulfated polysaccharide is a carrageenan.

35. The composition of claim 34 wherein said carrageenan is selected from the group consisting of iota carrageenan, kappa carrageenan, lambda carrageenan and mixtures thereof.

36. The composition of claim 33 wherein the pharmacologically active component is selected from the group consisting of decongestants, expectorants, antitussives, antihistamines, bronchodilators, demulcents, anti-inflammatory agents, antipyretics, analgesics, anesthetics, antimicrobials, antibiotics, peroxides, antibacterials, anticalculus agents, nutritional supplements, antacids, H2-receptor antagonists, laxatives, antidiarrheals, anorexiants, anticholinergics, antinauseants, and mixtures thereof.

37. The composition of claim 33 wherein said composition is selected from the group consisting of compositions for the treatment cough/cold symptoms, nutritional supplements and compositions for the treatment of gastrointestinal distress.

38. The composition of claim 36 wherein said composition is a nutritional supplement.

39. The composition of claim 38 wherein said nutritional supplement comprises at least one amino acid.

40. The composition of claim 39 wherein said at least one amino acid is selected from the group consisting of L-histidine, L-isoleucine, L-leucine, L-lysine, L-methionine, L-phenylalanine, L-threonine, L-tryptophan, L-tyrosine, L-valine and mixtures thereof.

41. The composition of claim 39 wherein the concentration of said at least one amino acid present in the pharmaceutical composition is at least 0.1 g per 100 g of said composition.

42. The composition of claim 41 wherein said carrageenan is present in a ratio of at least 0.5 parts carrageenan to 1 part amino acid present in the pharmaceutical composition.

43. The composition of claim 42 wherein said carrageenan is present in a ratio of at least 0.8 part carrageenan to 1 part amino acid present in the pharmaceutical composition.

44. The composition of claim 34 wherein said carrageenan compound is an iota carrageenan.

45. The composition of claim 34 wherein said carrageenan is kappa carrageenan.

46. The composition of claim 34 wherein said carrageenan is lambda carrageenan.

47. The composition of claim 33 which further comprises at least one element selected from the group consisting of nucleosides, nucleotides, antioxidant system, natural flavor, artificial flavor, sweetener, artificial sweetener, oil, fat major trace and ultratrace minerals, minerals, vitamins and m-inositol.

48. The composition of claim 39 wherein said amino acid is a mixture consisting of 4-8% L-histidine, 6-10% L-isoleucine, 10-15% L-leucine, 5-15% L-lysine, 5-20% L-methionine, 5-15% L-phenylalanine, 5-15% L-threonine, 1-8% L-tryptophan, 5-15% L-tyrosine, and 15-25% L-valine.

49. The composition of claim 48 further comprising at least one vitamin and at least one source of minerals.

50. The composition of claim 33 wherein said composition is in a dry powder form.

51. The composition of claim 33 wherein said composition is dissolved or dispersed in an aqueous based medium.

52. The composition of claim 24 wherein the composition is a beverage and comprises at least 1 g/l of at least one amino acid and at least 0.1 g/i of sulfated polysaccharide.

53. The composition of claim 52 further comprising at least one additional component selected from the group consisting of a flavor component, a sweetener, a mineral supplement, a fat, an acidulant, a buffer, a vitamin or a mixture thereof.

54. The composition of claim 24 wherein the composition is a fabricated food and comprises at least 1 g/l of at least one amino acid and at least 0.1 g/l of sulfated polysaccharide.

55. The composition of claim 54 further comprising at least one additional component selected from the group consisting of a flavor component, a sweetener, a mineral supplement, a fat, a fiber, a buffer, a vitamin or a mixture thereof.
